(19) United States
(12) Patent Application Publication
(10) Pub. No.: US 2016/0089312 A1
(43) Pub. Date: Mar. 31, 2016

(54) COSMETIC COMPOSITION CONTAINING AN OILY PHASE COMPRISING A SILICONE ELASTOMER IN DISPERSED AQUEOUS DROPLETS AND A PARTICULAR SURFACTANT

(71) Applicant: L'OREAL, Paris (FR)

(72) Inventors: Valérie DIQUE-MOUTON, Chevilly Larue (FR); Pascal ARNAUD, L'Hay Les Roses (FR)

(21) Appl. No.: 14/785,563

(22) PCT Filed: Apr. 17, 2014
(86) PCT No.: PCT/IB2014/060811

§ 371 Date: Oct. 19, 2015

(30) Foreign Application Priority Data
Apr. 19, 2013 (FR) 13 53579

(51) Int. Cl. A61K 8/06; A61K 8/894; A61Q 1/02; A61K 8/895; A61K 8/37; A61K 8/92; A61K 8/89; A61K 8/58

(52) U.S. Cl. CPC A61K 8/064; A61K 8/89; A61K 8/894; A61K 8/585; A61K 8/895; A61K 8/37; A61K 8/92; A61Q 1/02; A61K 2800/596; A61K 2800/594

(57) ABSTRACT

The invention relates to a cosmetic composition comprising at least an oily phase, the said oily phase comprising at least: (i) a silicone elastomer present in aqueous droplets which are dispersed in said oily phase, (ii) at least one particular surfactant chosen from: —a) silicone surfactants having a chemical structure comprising silicone side chains and hydrophilic side chains, of which the general formula (I) is: (I) —b) silicone surfactants having a chemical structure comprising hydrophilic chains at both ends (α- ω̅substitution) of the main chain, of general formula (II) below: (II) —c) hydrocarbon-based surfactants resulting from the esterification of a mixture of polyglycerol with (i) a polyhydroxystearic acid, with 2 to 5 polyglycerol units, (ii) linear or branched aliphatic dicarboxylic acids containing 4 to 14 carbon atoms, and (iii) saturated or unsaturated, linear or branched fatty acids containing from 16 to 20 carbon atoms.

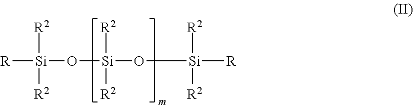

The invention relates to the field of care and/or makeup compositions for the skin. More specifically, the invention relates to compositions which produce, after application to the skin, a smoothing effect on the relief, in particular on the pores, wrinkles and fine lines.

The invention relates more specifically to a cosmetic composition comprising an oily phase containing at least (i) a silicone elastomer present in aqueous droplets which are dispersed in said oily phase and (ii) at least one particular surfactant.

Men and women, whatever their age, are concerned about the appearance of their skin, in particular facial skin. Facial skin may be marked by the presence of wrinkles and fine lines, blemishes, hyperpigmentation, shadows under the eyes, bags and loss of elasticity, but also by the presence of dilated and visible pores.

The appearance and/or visibility of the pores is a problem that it is sought to treat, in particular in the case of people with dilated pores, irrespective of their origin: ethnic (e.g. Asiatic population, Caucasian populations), excess sebum, ageing, loss of firmness, slackening, stress, fatigue, inappropriate hygiene, climatic factors, etc.

This skin imperfection in particular appears on the face and especially the T area (forehead, nose, cheeks and chin), in particular in the area of the nose and cheeks.

It is known practice to introduce silicone elastomers into cosmetic compositions, in particular care or makeup compositions, in order to obtain a matting effect or smoothing of the skin relief, in particular of the pores, wrinkles and fine lines.

This optical effect is desired, for example, in the case of makeup bases and foundations for hiding skin relief imperfections such as pores, wrinkles and fine lines.

In order to facilitate their use, these silicone elastomers are usually proposed by suppliers in the form of predispersions in volatile or non-volatile oils, which are introduced into the oily phases of the products during their manufacture.

However, for various reasons, the presence of the oil(s) used for conveying the elastomer in the predispersions is not always desired in the final composition: for example, this or these oil(s) may be detrimental to the matting and smoothing properties of the product and to its stability.

Furthermore, the proportion of these oils in the final composition may occasionally be high, especially if it is desired to introduce a large amount of elastomers into the composition and if the predispersion contains a small amount thereof.

There thus remains a need to obtain a dispersion of a silicone elastomer in an oily phase that is homogeneous and without the simultaneous addition of the oily vehicle for this elastomer.

It is also known practice to use silicone elastomers conveyed in the form of an aqueous suspension.

By way of example, a combination of a silicone elastomer coated with a silicone resin with a silicone elastomer conveyed in the form of an aqueous suspension, giving the product a novel elastic texture and a fresh sensation on application, is known from patent application FR 2 903 306. However, the illustrated products are only used as blushers.

U.S. Pat. No. 5,665,804 describes highly concentrated emulsions of silicone elastomers in water, known as “silicone latices” and also cosmetic compositions comprising these emulsions. The concentrated emulsions are then introduced directly into the oily phase of the compositions to gel them without using any surfactant.

However, the use of silicone elastomers in the form of aqueous suspensions does not always make it possible to obtain homogeneous dispersions.

In addition, there remains a need for compositions that can improve the smoothing effect on the skin relief and in particular on the pores.

There is also a need for compositions for obtaining satisfactory staying power and coverage and having a non-granular, smooth macroscopic appearance, and for affording a non-powdery, matt makeup that gives a soft feel.

The compositions according to the invention are directed towards satisfying these needs.

Thus, the technical problem underlying the present patent application was that of proposing compositions that can be applied easily and uniformly to the skin, with good spreading, and which optimally smooth out relief imperfections.

The cosmetic composition of the invention may advantageously be directed towards affording a homogeneous, matt, unifying and/or soft makeup effect.

It also allows a covering makeup to be obtained.

The Applicant has found, advantageously and unexpectedly, that it is possible to obtain a homogeneous dispersion of a silicone elastomer in an oily phase by using a silicone elastomer in the form of an aqueous suspension which is dispersed in the oily phase by means of particular surfactants.

This combination of a silicone elastomer introduced in the form of an aqueous suspension and of particular surfactants makes it possible to obtain homogeneous distribution of the silicone elastomer in any oily phase without the presence of an oily vehicle associated with the elastomer.

In addition, this combination makes it possible to obtain care or makeup products that have improved smoothing properties on skin relief imperfections, in particular the pores.

Compositions containing this combination apply easily and uniformly to the skin, without accumulating in the relief imperfections.

The present invention relates to a cosmetic composition comprising a physiologically acceptable medium containing at least one oily phase, the said oily phase comprising at least:

(i) a silicone elastomer in aqueous suspension,

(ii) at least one particular surfactant chosen from:
a) silicone surfactants having a chemical structure comprising silicone side chains and hydrophilic side chains, of which the general formula (I) is:

R¹ₐR²_{b}R³_{c}SiO_{(4-a-b-c)/2} (I)

a is greater than or equal to 1.0 and less than or equal to 2.5;

b is greater than or equal to 0.001 and less than or equal to 1.5;

c is greater than or equal to 0.001 and less than or equal to 1.5;

R¹ is an alkyl radical comprising from 1 to 10 carbon atoms, and R¹ is preferably a methyl radical,

R² is a hydrophilic group which may be represented by —CₘH₂ₘ—O—X

in which:

m is greater than 0 and less than or equal to 20;

X is:

[C₂H₄O]_{d}[C₃H₆O]ₑ R⁴ for which:

d is greater than or equal to 2 and less than or equal to 200;

e is greater than or equal to 0 and less than or equal to 200;

R⁴ is a hydrogen atom or an alkyl radical of 1 to 10 carbon atoms, preferably a hydrogen atom;

R³ is a silicone group which may be represented by: —C_{g}H_{2g}—(SiR₂O)ₕ—Si R₃ for which:

g is greater than or equal to 1 and less than or equal to 5;

h is greater than or equal to 1 and less than or equal to 500 and preferably greater than or equal to 1 and less than or equal to 50;

R is an alkyl radical of 1 to 10 carbon atoms;
b) silicone surfactants having a chemical structure comprising hydrophilic chains at both ends (α-ω substitution) of the main chain, of general formula (II) below:

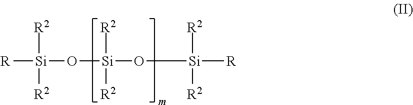

in which: R=—(CH₂)ₚO—(C₂H₄O)ₓ (C₃H₆O)_{y}R¹

in which:
R¹ represents H, CH₃ or CH₂CH₃,
p is an integer ranging from 1 to 5, x varies from 1 to 100, y varies from 0 to 50,
it being possible for the units (C₂H₄O) and (C₃H₆O) to be distributed randomly or in blocks,
the R² radicals represent a C₁-C₃ alkyl radical or a phenyl radical,
5≦m≦300,
c) hydrocarbon-based surfactants resulting from the esterification of a mixture of polyglycerol with (i) a polyhydroxystearic acid, with 2 to 5 polyglycerol units, (ii) linear or branched aliphatic dicarboxylic acids containing 4 to 14 carbon atoms, and (iii) saturated or unsaturated, linear or branched fatty acids containing from 16 to 20 carbon atoms.

By the expression “(i) a silicone elastomer in aqueous suspension” is more particularly meant that the silicone elastomer is present in aqueous droplets which are dispersed in said oily phase.

The said composition is advantageously a liquid composition. Advantageously, the composition according to the invention is a W/O or O/W emulsion, or alternatively a W/O/W or O/W/O triple emulsion.

Advantageously, the composition is a W/O or O/W/O emulsion.

A subject of the invention is also a process for making up and/or caring for the skin, comprising a step of applying a composition according to the invention.

The term “skin” is intended to denote all of the skin of the body, including the lips, and preferably the skin of the face, the neck and the neckline.

The invention also relates to a process for preparing a cosmetic composition according to the invention, comprising at least one step of dispersing into the oily phase an aqueous suspension of a silicone elastomer and at least one step of introducing at least one particular surfactant chosen from a), b) and c) as defined above into the oily phase.

These steps may be simultaneous or successive.

According to a preferred embodiment, the step of dispersing the aqueous suspension of silicone elastomer into the oily phase is performed after the step of introducing the particular surfactant into the oily phase.

According to a particular embodiment, the invention is directed towards a process for preparing a cosmetic composition according to the invention, comprising at least the following two steps (i) and (ii), in this order:

(i) preparing a W/O or W/O/W emulsion, in particular a W/O emulsion, by emulsification of an aqueous phase and oily phase(s), the outer oily phase containing at least one particular surfactant chosen from a), b) and c) as defined above, and

(ii) dispersing into the outer oily phase an aqueous suspension of a silicone elastomer.

Advantageously, the composition according to the invention comprises an emulsion consisting of an outer oily phase, which contains the particular surfactant.

The silicone elastomer, conveyed in the form of an aqueous suspension, is preferably introduced into the outer oily phase of the emulsion after emulsification.

Without wishing to be bound by any theory, it appears that introducing the silicone elastomer in aqueous suspension into the oily phase, which is preferably the outer phase, makes it possible to improve its activity relative to that observed in the prior art compositions in which it is in the aqueous phase.

The composition according to the invention has a particularly advantageous sensory effect, in particular in terms of homogeneity, mattness, softness, non-greasy appearance, smooth appearance and masking of pores, and is comfortable on application.

Silicone Elastomers in Dispersed Aqueous Droplets

The silicone elastomers conveyed in the form of an aqueous suspension which are used for the preparation of a composition according to the invention, in other words the silicone elastomers which are present in dispersed aqueous droplets in the final composition according to the invention, are partially or totally crosslinked organopolysiloxane elastomer particles.

The term “elastomer” means a supple, deformable material with viscoelastic properties and especially the consistency of a sponge or a supple sphere. This elastomer is formed from polymer chains of high molecular weight, the mobility of which is limited by a uniform network of crosslinking points.

The organopolysiloxane elastomers used in the composition according to the invention are partially or totally crosslinked and of three-dimensional structure. They may be chosen especially from the crosslinked polymers described in U.S. Pat. No. 5,928,660 from Dow Corning.

According to that patent, the crosslinked organopolysiloxane elastomer is obtained via addition and crosslinking reaction, in the presence of a catalyst in particular of platinum type, of at least:

(a) an organopolysiloxane (i) containing at least two vinyl groups in the α-ω position of the silicone chain per molecule; and

(b) an organosiloxane (ii) containing at least one hydrogen atom linked to a silicon atom per molecule.

The aqueous dispersion may especially be obtained as follows:

(a) mixing of an organopolysiloxane (i) containing at least two vinyl groups in the α-ω position of the silicone chain per molecule and of an organosiloxane (ii) containing at least one hydrogen atom linked to a silicon atom per molecule;

(b) adding a catalyst, in particular of platinum type;

(c) adding an aqueous phase containing an emulsifier to form an emulsion;

(d) polymerization of the organopolysiloxane (i) and of the organosiloxane (ii) in emulsion in the presence of a catalyst.

In particular, the organopolysiloxane (i) is chosen from n α,ω-dimethylvinylpolydimethylsiloxanes.

Reference may be made, for example, to L'Oréal patent application FR 2 800 605 for a detailed description of the process for manufacturing these aqueous suspensions of silicone elastomers.

The organopolysiloxane elastomer particles have a size ranging from 0.1 to 500 μm and better still from 3 to 200 μm. These particles may be spherical, flat or amorphous, and are preferably of spherical form.

These organosiloxane elastomer particles may be associated, in the aqueous suspension, with fatty substances, especially oils.

In addition, these organopolysiloxane elastomer particles are stably dispersed in water by means of one or more nonionic, cationic or anionic emulsifiers with an HLB of greater than or equal to 8. The proportion of emulsifiers in the aqueous suspension preferably ranges from 0.1 to 20 parts by weight per 100 parts by weight of the aqueous suspension of organopolysiloxane elastomer and better still from 0.5 to 10 parts by weight.

Thus, the silicone elastomer present in aqueous droplets dispersed in the oily phase of a composition of the invention is more particularly present in a form associated with one or more surfactant(s).

Of course, these surfactants are distinct from those so-called “particular surfactants” chosen from a), b) and c) surfactants that are separately introduced in the oily phase of the composition according to the invention.

According to a particular embodiment, the silicone elastomer and its surfactant(s) are associated in a dry weight ratio elastomer/surfactant(s) ranging from 30 to 1000.

By the expression “a form associated with” is meant that the silicone elastomer and its combined surfactant(s) are not present separately in the oily phase of the composition of the invention but are closely associated to each other.

For example, the association of said silicone elastomer with its surfactant(s) may be represented by a kind of entrapped form between silicone elastomer and surfactant(s), like the one that can be obtained at the end of an emulsion polymerization process for forming the silicone elastomer and that associate the so-formed silicone elastomer particles and the surfactant used for conducting the polymerization reaction.

Such associations of the silicone elastomer with its surfactant(s) exist also in some silicone elastomers that are commercialized in the form of an aqueous suspension or in a dry form (powder) as indicated in the INCI names of these products as described below.

The surfactants associated with the silicone elastomer in the dispersed aqueous droplets may preferably be non-ionic.

The preferred nonionic surfactants are polyoxyalkylenated alkyl ethers, polyoxyalkylenated sorbitan esters, polyoxyalkylenated esters, polyoxyalkylenated alkylphenyl ethers, ethoxylated amides, ethoxylated siloxanes and block copolymers of propylene oxide and ethylene oxide.

As nonionic surfactants associated with the silicone elastomer, mention may be made of the surfactants sold under the names Tergitol® TMN-6, Tergitol® 15S40, Tergitol® 15S3, Tergitol® 15S5 and Tergitol® 15S7 from Union Carbide Corporation (Danbury, Conn.) and Brij 30 and Brij 35 from ICI Chemicals (Wilmington, Del.); Triton® X405 from Röhm & Haas (Philadelphia, Pa.); Makon 10 from Stepan Company (Northfield, Ill.); Ethomid O/17 by Akzo Inc. (Chicago, Ill.); and Pluronic® F38 from BASF Corporation (Parsippany, N.J.).

The said nonionic surfactant is preferably a C₁₀-C₁₈ fatty alkyl polyoxyether, i.e. a polymer of polyethylene glycol (PEG) and of C₁₀-C₁₈ fatty acid(s), and is preferably a polymer of polyethylene glycol (PEG) and of C₁₂-C₁₆ fatty acid(s) comprising from 4 to 12 OE (oxyethylene) units. Examples of nonionic surfactants that may especially be mentioned include laureth-4, laureth-12 and C12-C14 pareth-12.

The aqueous suspensions of organopolysiloxane particles that can be used for the preparation of a composition according to the invention are, for example, those sold under the names BY 29-119, BY 29-122, BY 29-129 and DC 9509 by the company Dow Corning, the US INCI name of which is Dimethicone/vinyl dimethicone crosspolymer and C12-C14 pareth-12.

These suspensions comprise about 63% by weight of organopolysiloxane elastomer particles (and thus about 63% active material) relative to the total weight of the composition.

Mention may also be made of the reference Gransil LTX sold by the company Grant Industries, the INCI name of which is Cyclopentasiloxane (and) polysilicone-11 (and) water (and) laureth-4 and in which the content of organopolysiloxane elastomer particles is between 14% and 20%.

Finally, mention may be made of the reference Gransil EP-9, also sold by the company Grant Industries, the INCI name of which is Polysilicone-11 (and) water (and) laureth-12 (and) phenoxyethanol (and) ethylhexylglycerine and in which the content of non-volatile matter is between 59% and 62%.

The silicone elastomer in dispersed aqueous droplets is present in the composition of the invention in an active material amount ranging from 0.1% to 30% by weight, preferably from 0.5% to 20% by weight and even more preferentially from 1% to 10% by weight.

Particular Surfactants

The particular surfactants used in the present invention are chosen from three groups: a) silicone surfactants of formula (I), b) silicone surfactants of formula (II), and c) hydrocarbon-based surfactants resulting from the esterification of a mixture of polyglycerol with (i) a polyhydroxystearic acid with 2 to 5 polyglycerol units, (ii) aliphatic dicarboxylic acids, and (iii) fatty acids.

Group a) corresponds to the silicone surfactants of formula (I) described above. Preferably, the molecular weight of this silicone surfactant is between 500 and 200 000 g·mol⁻¹.

An example of a silicone surfactant of formula (I) that may be mentioned is PEG-9 polydimethylsiloxyethyl dimethicone sold under the reference KF 6028 by the company Shin-Etsu.

Group b) corresponds to the silicone surfactants of formula (II) described above.

Preferably, the α-ω-substituted oxyalkylenated silicone used according to the present invention corresponds to the general formula (II) for which all the R² radicals are methyl radicals, and:

R=—(CH₂)ₚO—(C₂H₄O)ₓ(C₃H₆O)_{y}R¹

in which:
R¹ represents H, CH₃ or CH₂CH₃,
p ranges from 2 to 4,
x ranges from 3 to 100,
y ranges from 0 to 50,
m ranges from 50 to 200.

More preferably, the average molecular weight of R ranges from 800 to 2600.

Preferably, the weight ratio of the C₂H₄O units relative to the C₃H₆O units ranges from 100/10 to 20/80. Advantageously, this ratio is around 42/58.

More preferably, R¹ is the methyl group.

According to a preferred embodiment of the group b), the α-ω oxyalkylenated silicone used corresponds to the following formula:

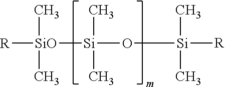

in which:
m=100,
R=(CH₂)₃—O—(C₂H₄O)ₓ—(C₃H₆O)_{y}—CH₃, where x ranges from 3 to 100, y ranges from 1 to 50, the weight ratio of the number of C₂H₄O to the number of C₃H₆O being around 42/58, the average molecular weight of R ranging from 800 to 1000.

As examples of products which may contain all or some of the α-ω-substituted oxyalkylenated silicones of group b) that can be used according to the invention, mention may especially be made of those sold under the names Abil EM 97 and Abil EM 97 S by the company Evonik Goldschmidt, or KF 6009, X22-4350, X22-4349 or KF 6008 by the company Shin-Etsu.

Group c) corresponds to the hydrocarbon-based surfactants resulting from the esterification of a mixture of polyglycerol with (i) a polyhydroxystearic acid, with 2 to 5 polyglycerol units (preferably 4 units), (ii) linear or branched aliphatic dicarboxylic acids containing 4 to 14 carbon atoms, preferably sebacic acid, and (iii) saturated or unsaturated, linear or branched fatty acids containing from 16 to 20 carbon atoms, preferably isostearic acid.

As a preferred example of a polyhydroxystearic acid ester of polyglycerol, mention may be made of polyglyceryl-4 diisostearate polyhydroxystearate sebacate of formula:

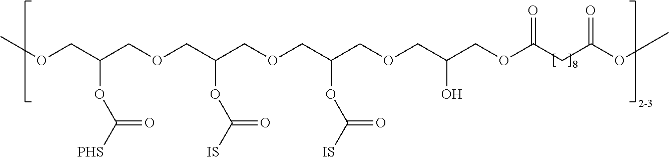

in which PHS denotes polyhydroxystearic acid and IS denotes isostearic acid.

Such a compound is prepared according to patent application US 2005/0 031 580 and sold under the name Isolan GPS by the company Evonik Goldschmidt.

According to the invention, the particular surfactant(s) are present in a content ranging from 0.1% to 15% by weight, preferably from 1% to 12% by weight and even more preferentially from 2% to 6% by weight relative to the total weight of the composition.

Oily Phase

The composition according to the invention comprises at least one oily phase, advantageously an outer oily phase.

The oily phase contains at least one oil. It may also contain other fatty substances.

Mention may be made, as oils which can be used in the composition of the invention, for example, of:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides, or alternatively, for example, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as Parleam oil;
fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils, for instance those described in document JP-A-2-295 912;
silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In the list of the abovementioned oils, the term “hydrocarbon-based oil” is understood to mean any oil predominantly comprising carbon and hydrogen atoms, and optionally ester, ether, fluoro, carboxylic acid and/or alcohol groups. Preferably, the oily phase(s) of the composition according to the invention comprise or even consist of a silicone oil.

The proportion of the oily phase(s) may range from 3% to 98% by weight, preferably from 10% to 90% by weight and preferably from 20% to 80% by weight relative to the total weight of the composition.

The other constituents that may be present in the oily phase are, for example, fatty acids, waxes, silicone resins and silicone elastomers.

These constituents may be chosen in a varied manner by a person skilled in the art so as to prepare a composition having the desired properties, for example in terms of consistency or texture.

Aqueous Phase

The emulsion according to the invention advantageously comprises at least one aqueous phase.

The aqueous phase comprises water. The water may be a floral water such as cornflower water and/or a mineral water such as Vittel water, Lucas water or La Roche Posay water and/or a spring water.

The water may be present in the composition according to the invention in a content ranging from 5% to 80% by weight, preferably ranging from 10% to 70% by weight and more preferentially ranging from 20% to 60% by weight relative to the total weight of the composition.

The aqueous phase may also comprise organic solvents that are miscible with water (at 25° C.), for instance primary alcohols such as ethanol and isopropanol, polyols such as glycerol, propylene glycol, butylene glycol, dipropylene glycol, diethylene glycols, glycol ethers and C1 to C4 alkyl ethers of mono-, di- or tripropylene glycol or of mono-, di- or triethylene glycol, and mixtures thereof.

The proportion of the aqueous phase(s) may range from 5% to 85% by weight and preferably from 20% to 70% by weight relative to the total weight of the composition.

Additional Emulsifiers

The emulsions may contain at least one additional emulsifier chosen from amphoteric, anionic, cationic and nonionic emulsifiers, used alone or as a mixture, and optionally a coemulsifier. The emulsifier and the coemulsifier are generally present in the composition in a proportion ranging from 0.3% to 30% by weight and preferably from 0.5% to 20% by weight relative to the total weight of the composition.

For the W/O emulsions, examples of emulsifiers that may be mentioned include dimethicone copolyols such as the mixture of cyclomethicone and of dimethicone copolyol sold under the name DC 5225 C by the company Dow Corning, and alkyl dimethicone copolyols such as the laurylmethicone copolyol sold under the name Dow Corning 5200 Formulation Aid by the company Dow Corning and the cetyl dimethicone copolyol sold under the name Abil EM 90® by the company Goldschmidt.

A crosslinked elastomeric solid organopolysiloxane comprising at least one oxyalkylene group, such as those obtained according to the procedure of Examples 3, 4 and 8 of document U.S. Pat. No. 5,412,004 and of the examples of document U.S. Pat. No. 5,811,487, especially the product of Example 3 (synthesis example) of U.S. Pat. No. 5,412,004, such as the product sold under the reference KSG 21 by the company Shin-Etsu, may also be used as surfactants for W/O emulsions.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; and mixtures thereof such as the mixture of glyceryl stearate and of PEG-40 stearate.

Additives

In a known manner, the cosmetic composition of the invention may also contain adjuvants that are common in the cosmetics field, such as hydrophilic or lipophilic gelling agents, preserving agents, solvents, fragrances, fillers, UV-screening agents, bactericides, odour absorbers, dyestuffs, plant extracts, salts, antioxidants, basic agents, acids or nonionic, anionic or cationic surfactants.

The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

Dyestuffs

The composition in accordance with the invention may also comprise at least one dyestuff.

Such a dyestuff may be chosen, for example, from water-soluble or water-insoluble, liposoluble or non-liposoluble, organic or mineral dyestuffs, especially of the type such as pigments or nacres, conventionally used in cosmetic compositions, materials with an optical effect, and mixtures thereof.

The dyestuffs may be present in a proportion of from 0.01% to 40% by weight, especially from 0.5% to 25% by weight or even from 1% to 15% by weight, relative to the total weight of the composition.

The term “pigments” should be understood as meaning white or coloured, inorganic (mineral) or organic particles, which are intended to colour the resulting film.

As inorganic pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

The pigment may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment with a structure that may be, for example, of the type such as silica microspheres containing iron oxide type. An example of a pigment having this structure is the product sold by the company Miyoshi under the reference PC Ball PC-LL-100 P, this pigment being constituted of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPPs) described in documents EP-A-542 669, EP-A-787 730, EP-A-787 731 and WO-A-96/08537.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superimposed at least two successive layers of metal oxides and/or of organic colorants.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orangey, brown, gold and/or coppery colour or tint.

As illustrations of nacres that may be used in the context of the present invention, mention may especially be made of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale G005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

A cosmetic composition according to the invention may also contain at least one material with a specific optical effect.

This effect is different from a simple conventional hue effect, i.e. a unified and stabilized effect as produced by standard dyestuffs, for instance monochromatic pigments.

For the purposes of the invention, the term “stabilized” means lacking an effect of variability of the colour as a function of the angle of observation or alternatively in response to a temperature change.

For example, this material may be chosen from particles with a metallic glint, goniochromatic colouring agents, diffractive pigments, thermochromic agents, optical brighteners, and also fibres, especially interference fibres. Needless to say, these various materials may be combined so as to afford the simultaneous manifestation of two effects, or even of a novel effect in accordance with the invention.

The particles with a metallic tint that can be used in the invention are in particular chosen from:
particles of at least one metal and/or of at least one metal derivative,
particles comprising a monomaterial or multimaterial organic or mineral substrate, at least partially coated with at least one layer with a metallic tint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are preferred metals.

The term “metal derivatives” is intended to denote compounds derived from metals, especially oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of copper metal powders or alloy mixtures such as the reference 2844 sold by the company Radium Bronze, metallic pigments such as aluminium or bronze, such as those sold under the name Rotosafe 700 from the company Eckart, the silica-coated aluminium particles sold under the name Visionaire Bright Silver from the company Eckart and metal alloy particles, for instance the silica-coated bronze (alloy of copper and zinc) powders sold under the name Visionaire Bright Natural Gold from the company Eckart.

They may also be particles comprising a glass substrate, such as those sold by the company Nippon Sheet Glass under the name Microglass Metashine.

The goniochromatic colouring agent may be chosen, for example, from multilayer interference structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company DuPont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Caribbean Blue by the company Merck. Mention may also be made of the Infinite Colors pigments from the company Shiseido. Different effects are obtained depending on the thickness and the nature of the various layers. Thus, with the Fe₂O₃/SiO₂/Al/SiO₂/Fe₂O₃ structure, the colour changes from greenish gold to reddish grey for SiO₂ layers of 320 to 350 nm; from red to gold for SiO₂ layers of 380 to 400 nm; from violet to green for SiO₂ layers of 410 to 420 nm; from copper to red for SiO₂ layers of 430 to 440 nm.

As examples of pigments with a polymeric multilayer structure, mention may be made of those sold by the company 3M under the name Color Glitter.

Examples of liquid-crystal goniochromatic particles that may be used include, for example, those sold by the company Chenix and also the products sold under the name Helicone® HC by the company Wacker.

The liposoluble dyes are, for example, Sudan red, DC Red 17, DC Green 6, β-carotene, soybean oil, Sudan brown, DC Yellow 11, DC Violet 2, DC Orange 5 and quinoline yellow.

The water-soluble dyes are, for example, beetroot juice or methylene blue.

According to one embodiment, an emulsion or a composition according to the invention may comprise at least one dyestuff chosen, for example, from organic dyestuffs and mineral dyestuffs, such as pigments and nacres, materials with a specific optical effect, and mixtures thereof.

According to another embodiment, an emulsion or a composition according to the invention is free of dyestuff, and may constitute a makeup base.

Fillers

The compositions of the invention may advantageously comprise at least one silicone elastomer powder coated with a silicone resin. The silicone elastomer, also known as an organopolysiloxane elastomer, is advantageously crosslinked and may be obtained via a crosslinking addition reaction of diorganopolysiloxane containing at least one hydrogen bonded to silicon and of diorganopolysiloxane containing ethylenically unsaturated groups bonded to silicon, especially in the presence of a platinum catalyst; or via a dehydrogenation crosslinking condensation reaction between a hydroxyl-terminated diorganopolysiloxane and a diorganopolysiloxane containing at least one hydrogen bonded to silicon, especially in the presence of an organotin compound; or via a crosslinking condensation reaction of a hydroxyl-terminated diorganopolysiloxane and of a hydrolysable organopolysilane; or via thermal crosslinking of organopolysiloxane, especially in the presence of an organoperoxide catalyst; or via crosslinking of organopolysiloxane by high-energy radiation such as gamma rays, ultraviolet rays or an electron beam.

Preferably, the elastomeric crosslinked organopolysiloxane is obtained by a crosslinking addition reaction (A2) of a diorganopolysiloxane containing at least two hydrogens each bonded to a silicon, and (B2) of a diorganopolysiloxane containing at least two ethylenically unsaturated groups bonded to silicon, especially in the presence (C2) of a platinum catalyst, for instance as described in patent application EP-A-295 886.

In particular, the organopolysiloxane may be obtained by reaction of dimethylvinylsiloxy-terminated dimethylpolysiloxane and of trimethylsiloxy-terminated methylhydropolysiloxane, in the presence of a platinum catalyst. The organopolysiloxane elastomer powder is coated with silicone resin.

According to a preferred embodiment, the silicone resin may be a silsesquioxane resin, as described, for example, in patent U.S. Pat. No. 5,538,793, the content of which is incorporated herein by way of reference.

The particles of silicone elastomer powder are preferably spherical with a mean size ranging from 0.1 to 500 μm, preferably from 3 to 200 μm and better still from 10 to 20 μm.

They may have a JIS-A hardness of less than or equal to 80 (especially ranging from 5 to 80) and preferably less than or equal to 65 (especially ranging from 5 to 65). The JIS-A hardness is measured according to the method JIS K 6301 (1995) established by the Japanese Industrial Standards Committee.

The silicone elastomer powders coated with a silicone resin used according to the invention are especially described in patent applications JP-A 61-194 009, EP-A 242 219, EP-A 295 886 and EP-A 765 656.

Such elastomer powders coated with silicone resin are especially sold under the names KSP-100, KSP-101, KSP-102, KSP-103, KSP-104 and KSP-105 by the company Shin-Etsu, and have the INCI name Vinyl dimethicone/methicone silsesquioxane crosspolymer.

The silicone elastomer powder coated with a silicone resin may be present in the composition in a content ranging from 1% to 20% by weight, preferably from 2% to 15% by weight and better still from 3% to 10% by weight relative to the total weight of the said composition.

The compositions of the invention may also advantageously comprise at least one filler based on polyalkyl (meth)acrylate, i.e. a filler which contains a polymer comprising at least the alkyl (meth)acrylate monomer.

The term “fillers based on polyalkyl (meth)acrylate” means fillers obtained by polymerization of an alkyl (meth)acrylate, in the form of a homopolymer or a copolymer.

By way of example, the filler which contains a polymer comprising at least the alkyl (meth)acrylate monomer is chosen from:
polymethyl methacrylate, especially crosslinked,
copolymers of methyl methacrylate and of butyl acrylate,
copolymers of methyl acrylate and of ethylene,
crosslinked copolymers of methyl methacrylate and of ethylene glycol bis(methacrylate), and
crosslinked copolymers of methyl methacrylate and of ethylene bis(methacrylate).

More particularly, mention may be made of:
the polymethyl methacrylate sold under the name SJ Touch 1 by the company Nihon Junyaku,
the methyl methacrylate/butyl acrylate copolymer sold under the name Sepipress M by the company SEPPIC,
the methyl acrylate/ethylene copolymer sold under the name EMAA by the company Kobo Products Inc.,
the methyl methacrylate/ethylene glycol bis(methacrylate) crosslinked copolymer sold under the name Ganzpearl GMP 0820 by the company Ganz Chemical, under the name Techpolymer MBP-8 by the company Sekisui Plastics or under the name SunPMMA-S by the company Sunjin Chemical,
the methyl methacrylate/ethylene bis(methacrylate) crosslinked copolymer sold under the name Ganzpearl PM 030 by the company Ganz Chemical,
the crosslinked polymethyl methacrylate sold under the name Covabead LH 85 by the company LC Wackherr.

The fillers consisting of crosslinked polymers are preferential.

These fillers are generally in the form of hollow or filled white spherical particles whose number-average size is generally on the micrometre scale, and in particular ranges from 3 to 20 microns and generally from 7 to 15 microns. The term “number-average size” denotes the dimension given by statistical particle size distribution for half the population, referred to as D50.

These fillers may be present in the composition in an amount ranging from 0.1% to 15% by weight, in particular from 0.5% to 10% and more particularly from 1% to 5% by weight relative to the total weight of the composition.

As fillers that may be used in the composition of the invention, examples that may also be mentioned, besides pigments, are matting agents, especially polyamide (Nylon®) powders, for instance Nylon 12 particles such as Orgasol from Arkema with a mean size of 10 microns and a refractive index of 1.54, and soft-focus fillers.

The soft-focus filler may be any material capable of modifying wrinkles via its intrinsic physical properties and of concealing them. These fillers may especially modify wrinkles via a tightening effect, a covering effect or a soft-focus effect.

The following compounds may be given as examples of fillers:
porous silica microparticles, for instance the silica beads SB150 and SB700 from Miyoshi with a mean size of 5 μm; the series-H Sunspheres from Asahi Glass, for instance Sunspheres H33, H51 with respective sizes of 3.5 and 5 μm;
hollow hemispherical particles of silicone resins, for instance NLK 500®, NLK 506® and NLK 510® from Takemoto Oil and Fat, described especially in EP-A-1 579 849;
silicone resin powders, for instance the silicone resin Tospearl® 145A from GE Silicone, with a mean size of 4.5 μm;
talc/titanium dioxide/alumina/silica composite powders, for instance the Coverleaf AR-80® products from the company Catalyst & Chemicals.

Cosmetic Active Agents

A person skilled in the art may also add one or more additional cosmetic active agent(s) as a function of the effect desired on the keratin materials.

The cosmetic active agents will be present in the composition according to the invention in a content ranging from 0.001% to 20% by weight relative to the total weight of the composition, preferably from 0.01% to 10%, even more preferentially from 0.5% to 5% and more preferably from 0.1% to 1% by weight relative to the total weight of the composition.

Advantageously, the composition of the invention may comprise an agent for reducing the visibility of pores.

The term “agent for reducing the visibility of pores” especially means an agent that is capable of reducing the appearance and/or visibility of pores, by tightening the pores and/or reducing the size and/or number of the visible pores; as an example of an agent for reducing the visibility of pores, mention may be made especially of C-glycoside derivatives.

Galenics

The compositions according to the invention comprise a physiologically acceptable medium, i.e. a non-toxic medium that can be applied to human skin, which is of pleasant appearance, odour and feel.

In particular, they will be cosmetic compositions for caring for and/or making up the skin, and more particularly facial skin.

More specifically, the makeup products may be of the type such as foundations, makeup bases, face powders, eyeshadows, concealer products or blushers, or alternatively a body makeup product or a skin colouring product, and especially foundations. Preferably, the composition according to the invention is a makeup base or a foundation.

The skincare product may be a composition for protecting, treating or caring for the face, for example a day cream, night cream, antisun composition, protective or care body milk or after-sun milk, and more preferably a facial care composition.

In addition, the compositions used according to the invention may be more or less fluid and may have the appearance of a white or coloured cream, an ointment, a milk or a paste. Preferentially, the compositions according to the invention are fluid.

According to a particular embodiment of the invention, the composition according to the invention contains a water-in-oil (W/O) or multiple (O/W/O) emulsion, and more preferably the composition is a water-in-oil (W/O) or multiple (O/W/O) emulsion.

Advantageously, the silicone elastomer introduced into the composition in aqueous suspension form is dispersed in the form of aqueous droplets in the outer oily phase.

Specifically, according to the preferred preparation process of the invention, the silicone elastomer conveyed in aqueous suspension is introduced into the outer oily phase of a W/O or O/W/O emulsion obtained previously by emulsification of an aqueous phase and an oily phase containing at least one particular surfactant, with stirring.

According to another aspect, the invention also relates to a cosmetic assembly comprising:

i) a container delimiting at least one compartment, the said container being closed by a closing member; and

ii) a composition as described previously, placed inside the said compartment.

The container may be of any suitable form. It may especially be a bottle, a tube, a jar, a case, a can, a sachet or a box.

The closing member may be in the form of a removable stopper, a lid, a cap, a tear-off strip or a capsule, especially of the type comprising a body attached to the container and a cover cap articulated on the body. It may also be in the form of a member for selectively closing the container, especially a pump, a valve or a flap valve.

The container may be combined with an applicator. The applicator may be in the form of a fine brush, as described, for example, in patent FR 2 722 380. The applicator can be in the form of a foam or elastomer pad, of a felt-tipped pen or of a spatula. The applicator may be free (powder puff or sponge) or securely fastened to a shaft borne by the closing member, as described, for example, in patent U.S. Pat. No. 5,492,426. The applicator may be solidly attached to the container, as described, for example, in patent FR 2 761 959.

The product may be contained directly in the container, or indirectly. By way of example, the product may be arranged on an impregnated support, especially in the form of a wipe or a pad, and arranged (individually or in plurality) in a box or in a sachet. Such a support incorporating the product is described, for example, in patent application WO 01/03538.

The closing member may be coupled to the container by screwing. Alternatively, the coupling between the closing member and the container is done other than by screwing, especially via a bayonet mechanism, by click-fastening, clamping, welding, adhesive bonding or by magnetic attraction. The term “click-fastening” in particular means any system involving the crossing of a bead or cord of material by elastic deformation of a portion, especially of the closing member, followed by return to the elastically unconstrained position of the said portion after the crossing of the bead or cord.

The container may be at least partially made of thermoplastic material. Examples of thermoplastic materials that may be mentioned include polypropylene or polyethylene.

The invention also relates to a cosmetic skin treatment process for reducing the appearance and/or visibility of pores, comprising the application to facial skin of the composition according to the invention.

The invention also relates to a cosmetic skin treatment process for improving the grain of the skin, in particular for tightening and/or refining the grain of the skin, comprising the application to facial skin of the composition according to the invention.

According to a particular embodiment, the composition is applied to the area of the face, in particular the T area (forehead, nose, cheeks and chin), in particular the cheeks and the nose.

According to another particular embodiment, the composition is applied to skin presenting visible or dilated pores.

According to an alternative, the composition is applied to greasy skin.

According to another alternative, the composition is applied to elderly skin.

The invention will now be illustrated by the non-limiting examples that follow.

### EXAMPLES

### Examples A to O

### Influence of the Nature of the Surfactant

Examples A to O show the influence of the structure of the surfactant on the dispersibility of the aqueous suspension of silicone elastomer (BY 29-119 from Dow Corning) in an oily phase.

| | | Mass % |
|---|---|---|
| A | Surfactant | 10.00 |
| | Isohexadecane | 27.00 |
| | Isododecane | 19.60 |
| | Dodecamethylpentasiloxane | 28.00 |
| B | Aqueous suspension of organopolysiloxane | 15.40 |
| | elastomer (63% elastomer) sold under the | (9.70 AM) |
| | reference BY 29-119 by the company Dow | |
| | Corning | |
| | TOTAL | 100% |

Procedure

The constituents of phase A are weighed out in the main beaker and are stirred with a Moritz blender (1600 rpm) while maintaining at room temperature.

Phase B is then added while stirring with the Moritz blender (3500 rpm) until homogeneous.

Results

| | | Appearance of the |
|---|---|---|
| | | dispersion (×10 |
| Example | Silicone surfactant | microscope) |
| Example A | PEG 9 polydimethylsiloxyethyl dimethicone | Homogeneous |
| (Invention) | sold under the reference KF 6028 by the | dispersion of the |
| | company Shin-Etsu | elastomer by |
| | | microscope |
| Example B | Bis-PEG/PPG-14/14 dimethicone sold under | Homogeneous |
| (Invention) | the reference Abil EM 97 by the company | dispersion of the |
| | Evonik Goldschmidt | elastomer by |
| | | microscope |
| Example C | Polyglyceryl-4 | Homogeneous |
| (Invention) | diisostearate/polyhydroxystearate/sebacate | dispersion of the |
| | sold under the reference Isolan GPS by the | elastomer by |
| | company Evonik-Goldschmidt | microscope |
| Example D | Lauryl PEG 9 polydimethylsiloxyethyl | Heterogeneous |
| (Comparative) | dimethicone sold under the reference KF | dispersion of the |
| | 6038 by Shin-Etsu | elastomer by |
| | | microscope (presence |
| | | of residual lumps) |
| Example E | PEG 10 dimethicone sold under the reference | Heterogeneous |
| (Comparative) | KF 6017 by Shin-Etsu | dispersion of the |
| | | elastomer by |
| | | microscope (presence |
| | | of residual lumps) |
| Example F | Cetyl PEG/PPG-10/1 dimethicone sold under | Heterogeneous |
| (Comparative) | the reference Abil EM 90 by the company | dispersion of the |
| | Evonik Goldschmidt | elastomer by |
| | | microscope (presence |
| | | of residual lumps) |
| Example G | Polyglyceryl-3 polydimethylsiloxyethyl | Heterogeneous |
| (Comparative) | dimethicone sold under the reference KF | dispersion of the |
| | 6104 by Shin-Etsu | elastomer by |
| | | microscope (presence |
| | | of residual lumps) |
| Example H | Lauryl polyglyceryl-3 | Heterogeneous |
| (Comparative) | polydimethylsiloxyethyl dimethicone sold | dispersion of the |
| | under the reference KF 6105 by Shin-Etsu | elastomer by |
| | | microscope (presence |
| | | of residual lumps) |
| Example I | Hydrogenated lecithin sold under the | Heterogeneous |
| (Comparative) | reference Nikkol Lecinol S 10 by the | dispersion of the |
| | company Nikko | elastomer by |
| | | microscope (presence |
| | | of residual lumps) |
| Example J | Polyglyceryl-3 diisostearate sold under the | Heterogeneous |
| (Comparative) | reference Lameform TGI by the company | dispersion of the |
| | Cognis | elastomer by |
| | | microscope (presence |
| | | of residual lumps) |
| Example K | Polyglyceryl-4 isostearate sold under the | Heterogeneous |
| (Comparative) | reference Isolan GI 34 by the company | dispersion of the |
| | Evonik-Goldschmidt | elastomer by |
| | | microscope (presence |
| | | of residual lumps) |
| Example L | Sorbitan isostearate sold under the reference | Heterogeneous |
| (Comparative) | N 120-[LO]-LQ-(MV) by the company | dispersion of the |
| | Croda | elastomer by |
| | | microscope (presence |
| | | of residual lumps) |
| Example M | Polyglyceryl-3 polyricinoleate sold under the | Heterogeneous |
| (Comparative) | reference Akoline PGPR by the company | dispersion of the |
| | Aarhus Karlshamn | elastomer by |
| | | microscope (presence |
| | | of residual lumps) |
| Example N | Polyglyceryl-6 polyricinoleate sold under the | Heterogeneous |
| (Comparative) | reference S-Face CR-1001 by the company | dispersion of the |
| | Sakamoto Yakuhin | elastomer by |
| | | microscope (presence |
| | | of residual lumps) |
| Example O | PEG-30 dipolyhydroxystearate sold under the | Heterogeneous |
| (Comparative) | reference Cithrol DPHS-SO-(MV) by the | dispersion of the |
| | company Croda | elastomer by |
| | | microscope (presence |
| | | of residual lumps) |

### Example 1

### Water-in-Oil (W/O) Foundation

This example illustrates the first family of silicone surfactants of the invention. PEG-9 polydimethylsiloxyethyl dimethicone (KF 6028 from Shin-Etsu) is thus used, which makes it possible to disperse the aqueous suspension of silicone elastomer (BY 29-119 from Dow Corning) in the outer oily phase of the emulsion.

| | | Example 1 |
|---|---|---|
| | | Mass % |
| A1 | PEG 9 polydimethylsiloxyethyl dimethicone sold under | 4.00 |
| | the reference KF 6028 by the company Shin-Etsu | |
| | Isohexadecane | 10.50 |
| | Isododecane | 7.55 |
| | Dodecamethylpentasiloxane | 7.50 |
| A2 | Dodecamethylpentasiloxane | 3.42 |
| | Pigments | 11 |
| A3 | Vinyl dimethicone/methicone silsesquioxane | 6.00 |
| | crosspolymer sold under the reference KSP 100 by the | |
| | company Shin-Etsu | |
| | PMMA sold under the reference Covabead LH 85 by the | 2.00 |
| | company Sensient | |
| B | Demineralized water | qs |
| | Glycerol | 5.00 |
| | Preserving agent | 0.50 |
| C | Aqueous suspension of organopolysiloxane elastomer | 6.00 |
| | (63% elastomer) sold under the reference BY 29-119 by | |
| | the company Dow Corning | |
| D | Absolute ethanol | 10.00 |
| | TOTAL | 100% |

### Procedure Example 1

The constituents of phase A1 are weighed out in the main beaker and are stirred with a Moritz blender (375 rpm) while maintaining at room temperature.

Phase A2 is prepared separately by milling three times in a three-roll mill the mixture of pigments and of dodecamethylpentasiloxane.

Next, phase A2 is added at room temperature, by stirring using a Moritz blender (375 rpm) until homogenized.

Phase A3 is then added, with continued stirring using a Moritz blender (500 rpm).

The aqueous phase B is also prepared separately, by weighing out in a beaker the glycerol and the preserving agent, and by adding water preheated to 95° C.

The aqueous phase is stirred using a magnetic bar until homogenized.

The emulsion is made at room temperature: the aqueous phase B is poured into the fatty phase while gradually increasing the stirring speed (Moritz blender) up to 1000 rpm. Stirring is continued for 10 minutes.

Phase C is then gradually added while increasing the stirring speed (Moritz blender) to 2000 rpm.

Finally, phase D is added with Moritz blender stirring (2600 rpm).

The product obtained is stirred using a Rayneri blender (paddles) for 15 minutes at 750 rpm.

Sensory Evaluation

A panel of 5 women from the laboratory, aged from 25 to 50 years old, was requested to use this foundation in a half-face makeup test.

It emerges from this evaluation that this foundation leads to a homogeneous, unifying makeup result and makes it possible to smooth out the pores.

### Example 2

### Water-In-Oil (W/O) Foundation

This example illustrates the second family of silicone surfactants of the invention.

Bis-PEG/PPG-14/14 Dimethicone (Abil EM 97 from Evonik Goldschmidt) is thus used, which makes it possible to disperse the aqueous suspension of silicone elastomer

(BY 29-119 from Dow Corning) in the outer oily phase of the emulsion.

| | | Example 2 |
|---|---|---|
| | | Mass % |
| A1 | Bis-PEG/PPG-14/14 dimethicone sold under the | 4.00 |
| | reference Abil EM 97 by the company Evonik | |
| | Goldschmidt | |
| | Isohexadecane | 10.50 |
| | Isododecane | 7.55 |
| | Dodecamethylpentasiloxane | 7.50 |
| A2 | Dodecamethylpentasiloxane | 3.42 |
| | Pigments | 11 |
| A3 | Vinyl dimethicone/methicone silsesquioxane | 6.00 |
| | crosspolymer sold under the reference KSP 100 by the | |
| | company Shin-Etsu | |
| | PMMA sold under the reference Covabead LH 85 by | 2.00 |
| | the company Sensient | |
| B | Demineralized water | qs |
| | Glycerol | 5.00 |
| | Preserving agent | 0.50 |
| C | Aqueous suspension of organopolysiloxane elastomer | 6.00 |
| | (63% elastomer) sold under the reference BY 29-119 | |
| | by the company Dow Corning | |
| D | Absolute ethanol | 10.00 |
| | TOTAL | 100% |

Procedure

Idem Example 1.

Sensory Evaluation

A panel of 5 women from the laboratory, aged from 25 to 50 years old, was requested to use this foundation in a half-face makeup test.

It emerges from this evaluation that this foundation leads to a homogeneous, unifying makeup result and makes it possible to smooth out the pores.

### Example 3

### Water-In-Oil (W/O) Foundation

This example illustrates the third family of surfactants of the invention. Polyglyceryl-4 diisostearate/polyhydroxystearate/sebacate (Isolan GPS from Evonik-Goldschmidt) is thus used, which makes it possible to disperse the aqueous suspension of silicone elastomer (BY 29-119 from Dow Corning) in the outer oily phase of the emulsion.

| | | Example 3 |
|---|---|---|
| | | Mass % |
| A1 | Polyglyceryl-4 | 4.44 |
| | diisostearate/polyhydroxystearate/sebacate sold under | |
| | the reference Isolan GPS by the company Evonik- | |
| | Goldschmidt | |
| | Isohexadecane | 11.67 |
| | Isododecane | 8.39 |
| | Dodecamethylpentasiloxane | 8.33 |
| A2 | Dodecamethylpentasiloxane | 3.80 |
| | Pigments | 11 |
| A3 | Vinyl dimethicone/methicone silsesquioxane | 6.67 |
| | crosspolymer sold under the reference KSP 100 by the | |
| | company Shin-Etsu | |
| | PMMA sold under the reference Covabead LH 85 by | 2.22 |
| | the company Sensient | |
| B | Demineralized water | qs |
| | Glycerol | 5.55 |
| | Preserving agent | 0.56 |
| C | Aqueous suspension of organopolysiloxane elastomer | 6.67 |
| | (63% elastomer) sold under the reference BY 29-119 | |
| | by the company Dow Corning | |
| | TOTAL | 100% |

Procedure

Idem Example 1 except that phase D is absent.

Sensory Evaluation

A panel of 4 women from the laboratory, aged from 25 to 50 years old, was requested to use this foundation in a half-face makeup test.

It emerges from this evaluation that this foundation leads to a homogeneous, unifying makeup result and makes it possible to smooth out the pores.

### Example 4

### In Accordance with the Invention

| | | Example 4 |
|---|---|---|
| | | Mass % |
| A1 | PEG 9 polydimethylsiloxyethyl dimethicone sold | 4.00 |
| | under the reference KF 6028 by the company Shin- | |
| | Etsu | |
| | Isohexadecane | 10.50 |
| | Isododecane | 7.55 |
| | Dodecamethylpentasiloxane | 7.50 |
| A2 | Dodecamethylpentasiloxane | 3.42 |
| | Pigments | 11 |
| A3 | Vinyl dimethicone/methicone silsesquioxane | 6.00 |
| | crosspolymer sold under the reference KSP 100 by | |
| | the company Shin-Etsu | |
| | PMMA sold under the reference Covabead LH 85 | 2.00 |
| | by the company Sensient | |
| B | Demineralized water | qs |
| | Glycerol | 5.00 |
| | Preserving agent | 0.50 |
| C | Aqueous suspension of organopolysiloxane | 6.00 |
| | elastomer sold under the reference Gransil EP-9 by | |
| | the company Grant Industries | |
| D | Absolute ethanol | 10.00 |
| | TOTAL | 100% |

Procedure

The constituents of phase A1 are weighed out in the main beaker and are stirred with a Moritz blender (375 rpm) while maintaining at room temperature.

Phase A2 is prepared separately by milling three times in a three-roll mill the mixture of pigments and of dodecamethylpentasiloxane.

Next, phase A2 is added at room temperature, by stirring using a Moritz blender (375 rpm) until homogenized.

Phase A3 is then added, with continued stirring using a Moritz blender (500 rpm).

The aqueous phase B is also prepared separately, by weighing out in a beaker the glycerol and the preserving agent, and by adding water preheated to 95° C.

The aqueous phase is stirred using a magnetic bar until homogenized.

The emulsion is made at room temperature: the aqueous phase B is poured into the fatty phase while gradually increasing the stirring speed (Moritz blender) up to 1000 rpm. Stirring is continued for 10 minutes.

Phase C is then gradually added while increasing the stirring speed (Moritz blender) to 2000 rpm.

Finally, phase D is added with Moritz blender stirring (2600 rpm).

The product obtained is stirred using a Rayneri blender (paddles) for 15 minutes at 750 rpm.

The foundation of Example 4 (invention) leads to a homogeneous, unifying and soft makeup result.

1. Cosmetic composition comprising a physiologically acceptable medium containing at least an oily phase, the said oily phase comprising at least:
(i) a silicone elastomer present in aqueous droplets which are dispersed in said oily phase, and
(ii) at least one particular surfactant chosen from:
a) silicone surfactants having a chemical structure comprising silicone side chains and hydrophilic side chains, of which the general formula (I) is:
R¹ₐR²_{b}R³_{c}SiO_{(4-a-b-c)/2} (I)
a is greater than or equal to 1.0 and less than or equal to 2.5;
b is greater than or equal to 0.001 and less than or equal to 1.5;
c is greater than or equal to 0.001 and less than or equal to 1.5;
R¹ is an alkyl radical comprising from 1 to 10 carbon atoms,
R² is a hydrophilic group which may be represented by —CₘH₂ₘ—O—X
in which:
m is greater than 0 and less than or equal to 20;
X is:
[C₂H₄O]_{d}[C₃H₆O]ₑ R⁴ for which:
d is greater than or equal to 2 and less than or equal to 200;
e is greater than or equal to 0 and less than or equal to 200;
R⁴ is a hydrogen atom or an alkyl radical of 1 to 10 carbon atoms;
R³ is a silicone group which may be represented by:
—C_{g}H_{2g}—(SiR₂O)ₕ—Si R₃ for which:
g is greater than or equal to 1 and less than or equal to 5;
h is greater than or equal to 1 and less than or equal to 500;
R is an alkyl radical of 1 to 10 carbon atoms;
b) silicone surfactants having a chemical structure comprising hydrophilic chains at both ends (α-ω substitution) of the main chain, of general formula (II) below: 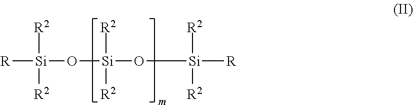
in which: R=—(CH₂)ₚO—(C₂H₄O) (C₃H₆O)_{y}R¹ in which:
R¹ represents H, CH₃ or CH₂CH₃,
p is an integer ranging from 1 to 5, x varies from 1 to 100, y varies from 0 to 50,
it being possible for the units (C₂H₄O) and (C₃H₆O) to be distributed randomly or in blocks,
the R² radicals represent a C₁-C₃ alkyl radical or a phenyl radical,
5≦m≦300,
c) hydrocarbon-based surfactants resulting from the esterification of a mixture of polyglycerol with (i) a polyhydroxystearic acid, with 2 to 5 polyglycerol units, (ii) linear or branched aliphatic dicarboxylic acids containing 4 to 14 carbon atoms, and (iii) saturated or unsaturated, linear or branched fatty acids containing from 16 to 20 carbon atoms.

2. Composition according to claim 1, in the form of a W/O or O/W/O emulsion.

3. Composition according to claim 1, in which the silicone elastomer in dispersed aqueous droplets is a partially or totally crosslinked elastomeric organopolysiloxane particle.

4. Composition according to claim 1, in which the silicone elastomer in dispersed aqueous droplets is present in the composition in an active material amount ranging from 0.1% to 30% by weight.

5. Composition according to claim 1, in which the particular surfactant is PEG-9 polydimethylsiloxyethyl dimethicone.

6. Composition according to claim 1, in which the particular surfactant is a silicone surfactant of general formula (II) for which:
all the radicals R² represent methyl radicals;
R=—(CH₂)ₚO—(C₂H₄O)ₓ(C₃H₆O)_{y}R¹
in which:
R¹ represents H, CH₃ or CH₂CH₃,
p ranges from 2 to 4,
x ranges from 3 to 100,
y ranges from 0 to 50, and
m ranges from 50 to 200.

7. Composition according to claim 1, in which the particular surfactant corresponds to the following formula: 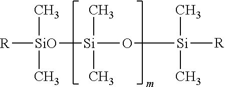
in which:
m=100, and
R=—(CH₂)₃—O—(C₂H₄O)ₓ—(C₃H₆O)_{y}—CH₃, where x ranges from 3 to 100, y ranges from 1 to 50, the weight ratio of the group C₂H₄O to the group C₃H₆O being about 42/58, the mean molecular weight of R ranging from 800 to 1000.

8. Composition according to claim 1, in which the particular surfactant is a hydrocarbon-based surfactant, which is polyglyceryl-4 diisostearate polyhydroxystearate sebacate of formula: 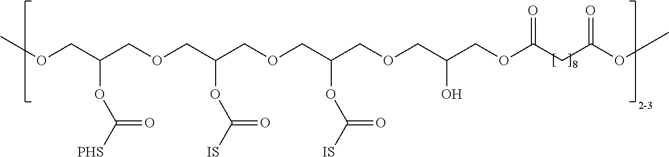
in which PHS denotes polyhydroxystearic acid and IS denotes isostearic acid.

9. Composition according to claim 1, in which the particular surfactant(s) are present in a content ranging from 0.1% to 15% by weight relative to the total weight of the composition.

10. Composition according to claim 1, in which the oily phase(s) comprise or even consist of a silicone oil.

11. Composition according to claim 1, in which the proportion of the oily phase(s) is from 3% to 98% by weight relative to the total weight of the composition.

12. Composition according to claim 1, in the form of a foundation, a makeup base, a face powder, an eyeshadow, a concealer product, a blusher or a body makeup product or skin colouring product.

13. Process for preparing a cosmetic composition according to claim 1, comprising at least one step of dispersing into the oily phase an aqueous suspension of a silicone elastomer and at least one step of introducing at least one particular surfactant chosen from a), b) and c) as defined in claim 1 into the oily phase.

14. Process for preparing a cosmetic composition according to claim 1, said composition being in the form of a W/O or O/W/O emulsion, comprising at least the following two steps (i) and (ii), in this order:
(i) preparing a W/O or O/W/O emulsion by emulsification of an aqueous phase and oily phase(s), the outer oily phase containing at least one particular surfactant chosen from a), b) and c) as defined in claim 1, and
(ii) dispersing into the outer oily phase an aqueous suspension of a silicone elastomer.

15. Process for making up and/or caring for the skin, comprising a step of applying a composition according to claim 1.
